(19) United States
(12) Patent Application Publication
(10) Pub. No.: US 2011/0178385 A1
(43) Pub. Date: Jul. 21, 2011

(54) PHOTOACOUSTIC MEASURING APPARATUS WITH MOVABLE DETECTOR ARRAY

(75) Inventors: Kazuhiko Fukutani, Yokohama-shi (JP); Takao Nakajima, Kawasaki-shi (JP)

(73) Assignee: CANON KABUSHIKI KAISHA, Tokyo (JP)

(21) Appl. No.: 13/121,899

(22) PCT Filed: Oct. 1, 2009
(86) PCT No.: PCT/JP2009/005083

§ 371 Date: Mar. 30, 2011

(30) Foreign Application Priority Data
Oct. 3, 2008 (JP) 2008-258569

(51) Int. Cl. A61B 6/00

(52) U.S. Cl. 600/407

(57) ABSTRACT

A measuring apparatus includes an acoustic transducer with a plurality of elements, each element detecting an acoustic wave generated from a sample and converting the wave into an electric signal; a movement control unit which moves the acoustic transducer from a first position to a second position; and a processing unit which generates image data on the basis of the electric signal. The acoustic transducer has a gap in the arrangement of the elements. The acoustic transducer detects an acoustic wave at the first position, is moved by the movement control unit such that the position of the gap at the first position corresponds to the position of the element at the second position, and then detects an acoustic wave at the second position. The processing unit generates image data on the basis of electric signals obtained at the first and second positions.

### TECHNICAL FIELD

The present invention relates to a measuring apparatus.

### BACKGROUND ART

In medical fields, imaging devices with X-ray, ultrasound, and magnetic resonance imaging (MRI) have been typically used. Also, the studies of optical imaging have positively progressed in the medical fields. The optical imaging obtains biological information by irradiating a sample such as a living body with light from a light source such as a laser, causing the light to propagate in the sample, and detecting the propagating light or the like. An example of such optical imaging techniques may be photoacoustic tomography (PAT).

The photoacoustic tomography is a technique which irradiates a sample with pulsed light generated from a light source, detects temporal profiles of acoustic waves generated from body tissues which have absorbed energy of the light propagating and being dispersed in the sample, at a plurality of positions surrounding the sample, mathematically analyses obtained signals, and visualizes information relating to optical characteristic values of the inside of the sample. Accordingly, an initial pressure distribution or an absorbed optical energy distribution, in particular, an absorbed optical energy distribution generated because of the light irradiation in the sample, can be obtained. The distribution can be used, for example, for specifying the position of a malignancy.

In general, with the photoacoustic tomography, an initial pressure distribution generated because of the light irradiation can be theoretically completely visualized as long as the temporal profiles of the acoustic waves can be measured by using an ideal acoustic transducer (wide band, point detection) on a surface of an enclosed space surrounding an entire sample, in particular, at various points on a spherical measurement surface. However, regarding an actual sample, it is difficult to obtain acoustic-wave detection information from the entire surface of the enclosed space surrounding the entire sample. In light of this, in some cases, a planar measurement system shown in FIG. 1 may be used. Referring to FIG. 1, reference numeral 1 denotes an acoustic transducer, 2 denotes a light absorber serving as an acoustic wave source, 3 denotes a sample, 4 denotes an image reconstruction region, and 5 denotes an acoustic wave. In such a plate measurement system, it has been mathematically known that an acoustic-wave-source distribution can be substantially completely reconstructed as long as the acoustic wave can be measured in a sufficiently large region (ideally, infinite surface), for the image reconstruction region 4 in which an initial pressure distribution generated because of pulsed light irradiation is visualized (see Physical Review E71, 016706,2005).

### CITATION LIST

Non Patent Literature

NPL 1: Physical Review E71, 016706, 2005

### SUMMARY OF INVENTION

When the size of an acoustic transducer 1 is increased and the number of elements included in the acoustic transducer 1 is increased so as to expand a measurement region of acoustic waves, an electronic control system for control of the acoustic transducer 1 becomes large, resulting in the electronic control system being an extremely expensive system. When an acoustic transducer with a large number of elements is to be produced, the acoustic transducer is divided into a plurality of element groups for easier fabrication, and the plurality of element groups are arranged. Consequently a large acoustic transducer is produced.

When the number of elements is a significantly large number, since a wiring cable for transmitting electric signals of the elements to the outside is restricted (or a cable may be increased in diameter), wiring may not be provided for some of the elements. Furthermore, a groove (boundary portion) is provided to reduce cross talk which is generated between the divided element groups. No acoustic wave is detected in the region occupied by the groove.

Accordingly, the present invention provides a measuring apparatus capable of generating image data, the finally obtained image data of which is more approximate to an actual acoustic-wave-source distribution although the size and the number of elements simultaneously detectable in an acoustic transducer are restricted.

A measuring apparatus according to an aspect of the present invention includes an acoustic transducer in which a plurality of elements are arranged, each element configured to detect an acoustic wave generated from a sample and convert the detected acoustic wave into an electric signal; a movement control unit configured to move the acoustic transducer from a first position to a second position; and a processing unit configured to generate image data on the basis of the electric signal. The acoustic transducer has a gap in the arrangement of the elements. The acoustic transducer detects an acoustic wave at the first position, is moved by the movement control unit such that the position of the gap at the first position corresponds to the position of the element at the second position, and then detects an acoustic wave at the second position. The processing unit generates image data on the basis of an electric signal obtained at the first position and an electric signal obtained at the second position.

With the aspect of the present invention, the measuring apparatus can be provided, which can generate image data more approximate to the actual acoustic-wave-source distribution although the size and the number of elements simultaneously detectable in the acoustic transducer are restricted.

### BRIEF DESCRIPTION OF DRAWINGS

FIG. 1 schematically illustrates an example configuration of a measuring apparatus according to related art.

FIG. 2 schematically illustrates an example configuration of a measuring apparatus according to an embodiment of the present invention.

FIG. 3 schematically illustrates an example configuration of an acoustic transducer of the measuring apparatus according to the embodiment.

FIG. 4 illustrates an example method of moving the acoustic transducer of the measuring apparatus according to the embodiment.

FIG. 5A illustrates an example of an acoustic wave source.

FIG. 5B illustrates an example image which is obtained without the acoustic transducer being moved.

FIG. 5C illustrates an example image which is obtained by the measuring apparatus according to the embodiment.

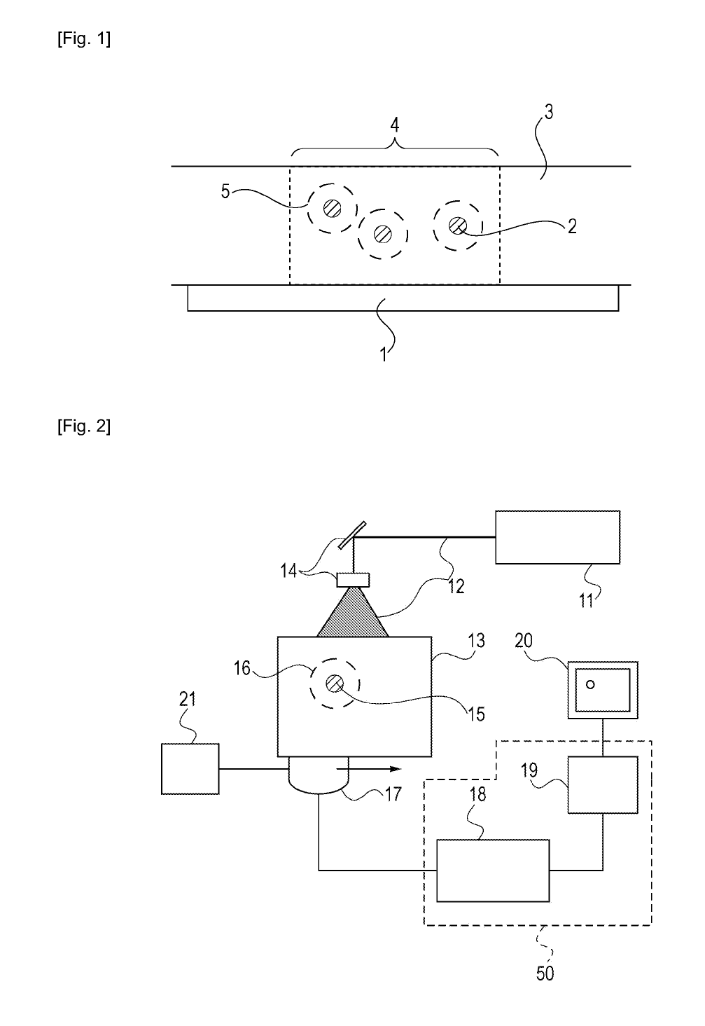

### DESCRIPTION OF EMBODIMENT

A measuring apparatus according to an embodiment of the present invention will be described below with reference to the drawings. The measuring apparatus of this embodiment can visualize biological information mainly for diagnosis of a malignancy or a vascular disease, or observation for the follow-up of a chemical treatment. Herein, the biological information is an acoustic-wave-source distribution. In particular, the biological information may be an initial pressure distribution in a living body or an absorbed optical energy distribution derived from the initial pressure distribution. Also, the biological information may be a chromophore concentration of a chromophore defining a body tissue, obtained from either of above-mentioned information. For example, the chromophore concentration may be an oxygen saturation.

Referring to FIG. 2, the measuring apparatus of this embodiment includes a light source 11 which irradiates a sample 13 with light 12, an optical component 14 such as a lens which guides the light 12 from the light source 11 to the sample 13, an acoustic transducer 17 which detects an acoustic wave 16 and converts the acoustic wave 16 into an electric signal, the acoustic wave 16 being generated because a light absorber 15 such as a blood vessel absorbs part of energy of the light 12, a processing unit 50 which processes the electric signal and generates image data, and a movement control system 21 which controls movement of the acoustic transducer 17. The processing unit 50 includes an electronic control system 18 for amplification and digital conversion, and an image reconstruction unit 19 such as a PC which generates image data (or reconstructs an image) on the basis of the electric signal after the digital conversion. The image data generated by the image reconstruction unit 19 is displayed as an image by a display device 20 such as a display.

By converting the light 12 into pulsed light and emitting the pulsed light to the sample 13, the acoustic wave 16 is generated from the light absorber 15 in the living body. This is because the temperature of the light absorber 15 is increased by the absorption of the pulsed light, the volume of the light absorber 15 is increased by the increase of the temperature, and hence the acoustic wave is generated. In this case, an optical pulse may have a pulse width which is capable of satisfying a confinement condition of heat and stress so that the light absorber 15 efficiently confines the absorption energy. Typically, the temporal ranges from about several nanoseconds to several tens of nanoseconds. Also, the acoustic transducer 17 can detect the acoustic wave 16 at various positions while the acoustic transducer 17 is mechanically moved by a movement control system 21.

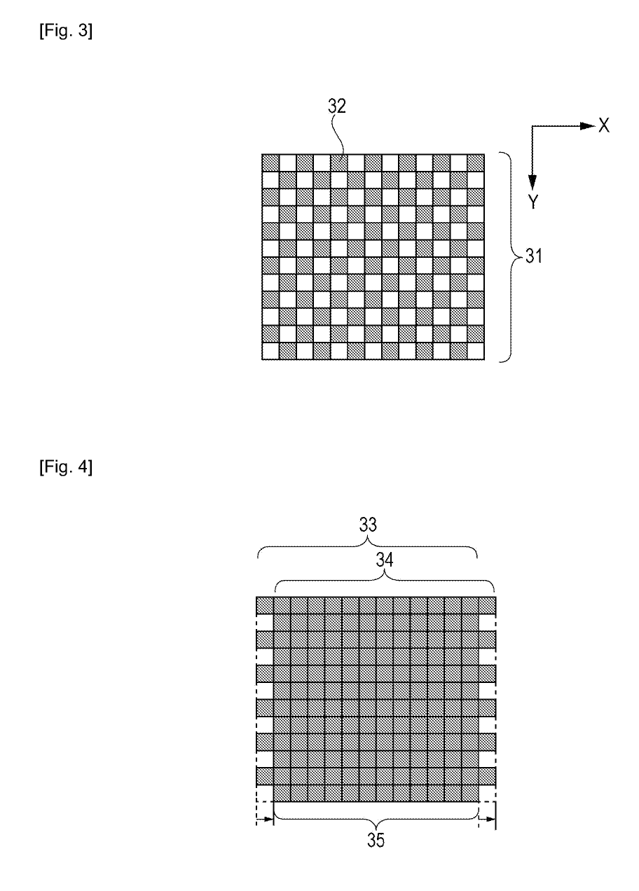

Now, a movement control method of the acoustic transducer 17 in the measuring apparatus according to this embodiment will be described below. FIG. 3 schematically illustrates an example of the acoustic transducer 17 shown in FIG. 2, when viewed from a surface being in contact with the sample 13.

In FIG. 3, reference numeral 31 denotes the entire acoustic transducer, and 32 denotes an element. The acoustic transducer 31 in FIG. 3 has elements 32 arranged in a staggered manner (i.e., elements and gaps being alternately arranged). When the acoustic transducer 31 with such element arrangement is moved in a movement direction (X direction) by a distance corresponding to a width of an element, the number of elements becomes apparently equivalent to the number of elements which are arranged without a gap, as shown in FIG. 4. Herein, a gap is a region in which an acoustic wave is not transmitted as an electric signal to the electronic control system 18. An element, which is not electrically connected, is also considered as a gap. That is, an element, which is apparently arranged in the acoustic transducer but is not electrically connected, cannot transmit an electric signal to the electronic control system 18. Thus, such an element is a gap. Referring to FIG. 4, reference numeral 33 denotes a detection region of the acoustic transducer before the movement (first position), and 34 denotes a detection region of the acoustic transducer after the movement (second position). Reference numeral 35 denotes a region in which the acoustic-wave detection region after the movement of the acoustic transducer is overlapped with the acousticwave detection region before the movement of the acoustic transducer.

The elements do not have to be arranged in a staggered manner. For example, elements and gaps may be alternately arranged line by line. The arrangement is not particularly limited as long as the number of elements is equivalent to the number of elements which are arranged without a gap. Namely, the arrangement of the elements may be any form as long as the positions of the gaps before the movement (first position) correspond to the positions of the elements after the movement (second position).

To easily generate image data (to easily reconstruct an image), a size of the gap may be an integral multiple of a size of the element, and a movement width of the acoustic transducer may be an integral multiple of the size of the element (width of the element in the movement direction).

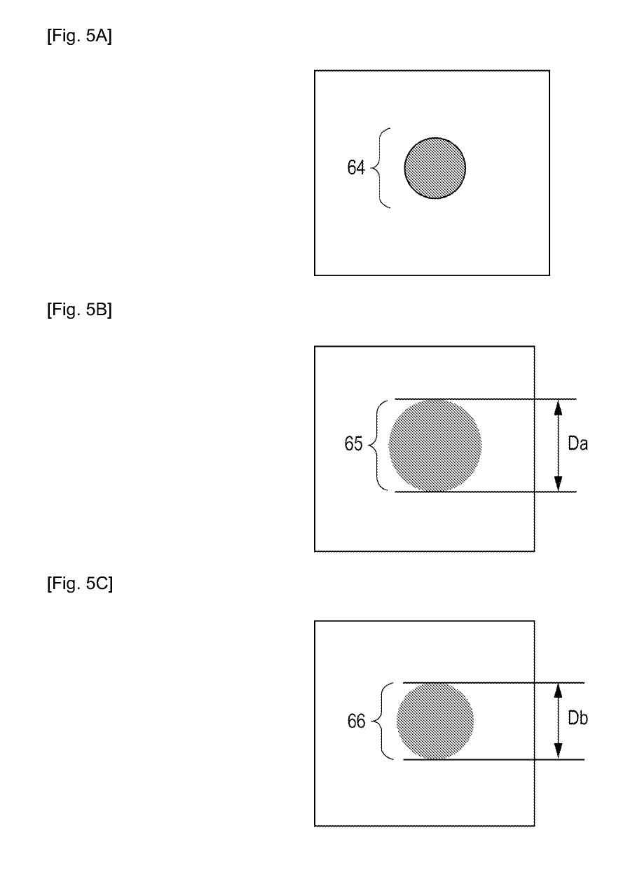

FIG. 5A illustrates an example of an initial acoustic-wave-source distribution on the acoustic transducer. Reference numeral 64 denotes an acoustic wave source. The acoustic transducer shown in FIG. 3 detects an acoustic wave generated from the acoustic wave source 64 without the movement control of the acoustic transducer. Then, image data is generated (i.e., an image is reconstructed) by using a typical image reconstruction method, such as a time domain algorism or a Fourier domain algorism. FIG. 5B provides a conceptual diagram of the generated (reconstructed) image. FIG. 5B shows the shape of an acoustic wave source 65 after the image reconstruction. Reference character Da is a diameter of the acoustic wave source 65 after the image reconstruction. FIG. 5C is a conceptual diagram showing an image which is reconstructed by moving the acoustic transducer in FIG. 3, by a distance corresponding to an element and using information of an acoustic wave before the movement and information of an acoustic wave after the movement. In this case, the image reconstruction method may be the time domain method or the Fourier domain method. The data before the movement and the data after the movement are merged and treated as information in association with a measurement element position. Thusly, the image reconstruction is carried out. FIG. 5C shows the shape of an acoustic wave source 66 after the image reconstruction, and Db is a diameter of the acoustic wave source 66 after the image reconstruction. Comparing the acoustic wave source 65 with the acoustic wave source 66, the acoustic wave source 66 is reconstructed more approximately to the shape of the acoustic wave source 64. Also, the diameter Db of the acoustic wave source 66 is smaller than the diameter Da of the acoustic wave source 65. This is because the acoustic transducer can be moved by the distance corresponding to the width of the element so that the acoustic-wave detection region before the movement is overlapped with the acoustic-wave detection region after the movement and hence the apparent number of elements to be used for the generation of the image data can be increased (i.e., signals similar to those of the acoustic transducer without a gap can be input). Consequently, the accuracy of the image reconstruction is increased, and imaging of the position and size of the acoustic wave source can be highly accurately performed although the size and number of the elements of the acoustic transducer are restricted.

When a plurality of small element groups are arranged to form a large acoustic transducer, a boundary portion may be provided between the small element groups. In the boundary portion, an acoustic wave cannot be detected. However, by defining the boundary portion as a gap portion, the size of the boundary portion may be increased, and the manufacturing can be facilitated.

In addition, when an acoustic transducer is configured such that a light source is arranged at a gap portion by using, for example, an optical fiber, the gap portion is easily irradiated with light from the acoustic transducer. In a case where a sample is irradiated with light and an acoustic wave is generated, when the light is emitted from the outside of the acoustic transducer, if the acoustic transducer is large, it is difficult to cause the light to propagate to a position directly below the acoustic transducer, resulting in image quality being degraded. In contrast, as long as the light is emitted in the detection elements of the acoustic wave, the light can be emitted to the position directly below the acoustic transducer, thereby increasing the quality of the reconstructed image.

The embodiment will be more specifically described below.

Referring to FIG. 2, the light source 11 emits light with a specific wavelength, which is absorbed by a characteristic component included in components of a living body. The light source may be integrally provided with the measuring apparatus, or may be provided separately. The light source 11 includes at least a pulsed light source which can generate pulsed light with the order ranging from several nanoseconds to several hundreds of nanoseconds. If a sound pressure of an acoustic wave to be detected may be small, light such as a sine wave with a variable intensity may be used instead of the pulsed light with the above-mentioned order. The light source 11 may be a laser of a large output; however, the light source 11 may use a light emitting diode instead of the laser. The laser may be a solid laser, a gas laser, a dye laser, a semiconductor laser, etc.

In this embodiment, the number of the light source 11 is one. However, a plurality of light sources may be used. In this case, to increase the irradiation intensity of light to be emitted on a living body, a plurality of light sources may be used, which oscillate light with a uniform wavelength. Alternatively, a plurality of light sources may be used, which oscillate light with different wavelengths to measure a difference in absorbed optical energy distributions as a result of the difference in the wavelengths. When the light source 11 uses a dye capable of converting a wavelength to be oscillated, an optical parametric oscillator (OPO), or a crystal of titanium sapphire or alexandrite, the difference in the absorbed optical energy distributions as a result of the difference in the wavelengths can be measured. Regarding the wavelength of the light source to be used, the wavelength may be in a range of from 700 to 1100 nm, the wavelength in the range being less absorbed by a living body. In a case where the absorbed optical energy distribution of a body tissue located relatively close to the surface of a living body is to be obtained, for example, light in a wavelength range of from 400 to 1600 nm may be used, the range being wider than the aforementioned wavelength range.

In FIG. 2, light 12 is emitted from the light source 11. The light 12 may propagate by using a light guide or the like. Though not shown, the light guide may be an optical fiber. When the optical fiber is used, a plurality of optical fibers may be used respectively for light sources, and the light may be guided to the surface of the living body. Alternatively, light of the plurality of light sources may be guided to a single optical fiber, and light of all light sources may be guided to the living body through the single optical fiber. The optical component 14 may be a mirror which mainly reflects light, and a lens which condenses light, enlarges light, or shapes light. The optical component 14 is not particularly limited as long as the light 12 emitted from the light source 11 and having a desired shape is emitted on the sample 13. Typically, light may be diffused by a lens to have a certain area rather than being condensed by a lens. The light irradiation region on the sample may be movable. In particular, the measuring apparatus of this embodiment may be configured such that the light emitted from the light source is movable on the sample. Accordingly, the light can be emitted in a wide area. In addition, the light irradiation region on the sample (light to be emitted on the sample) may be moved in synchronization with the acoustic transducer. The light irradiation region on the sample may be moved by using the movable mirror, or by mechanically moving the light source.

Since this embodiment is aimed at diagnosis of a malignancy or a vascular disease, or observation for the follow-up of a chemical treatment of a human body or an animal body, the sample 13 may be, for example, any of subjects for the diagnosis, such as a breast, a finger, a hand, or a leg of a human body or an animal body. A light absorber of the sample 13 may be a part with a large absorption coefficient in the sample 13. For example, when a human body is a subject of measurement, the light absorber may be hemoglobin, a blood vessel containing hemoglobin by a large amount, and a malignancy containing a new blood vessel.

The acoustic transducer 17 in FIG. 2 detects an acoustic wave generated from a chromophore which has absorbed part of energy of light propagating in the sample, and converts the acoustic wave into an electric signal. The acoustic transducer of the present invention may be any type of acoustic transducer, such as a transducer using piezoelectric phenomenon, a transducer using optical resonation, or a transducer using variation in volume, as long as the acoustic transducer can detect an acoustic wave.

The acoustic transducer provided in the measuring apparatus of this embodiment may be configured such that the elements are two-dimensionally arranged as shown in FIG. 3. With the two-dimensionally arranged elements, acoustic waves can be detected simultaneously at a plurality of positions. Accordingly, the detection time can be decreased, and the influence of vibration of a sample can be reduced. Though not shown, an acoustic impedance matching agent, such as gel or water, may be arranged between the acoustic transducer 17 and a sample to decrease reflection of acoustic waves.

The movement control system 21 of the acoustic transducer 17 in FIG. 2 uses a driving stage with a normal motor etc. and a stage controller. However, it is not limited thereto as long as the acoustic transducer 17 can be two-dimensionally operated.

The acoustic transducer of this embodiment is positioned and moved by a step-and-repeat method in which rest, detection, and movement are repeatedly performed. The acoustic transducer detects an acoustic wave in a stop state. The reception of the acoustic wave in the stop state at one position may be repeated a plurality of times. A plurality of received signals may be averaged, and the average value may be used. Accordingly, image data with reduced noise can be generated.

The electronic control system 18 in FIG. 2 amplifies an electric signal obtained by the acoustic transducer 17, and converts the obtained electric signal by analog-to-digital conversion. The image reconstruction unit 19 in FIG. 2 may be any configuration as long as the configuration can store data obtained from the electronic control system 18, and convert the data into image data of an absorbed optical energy distribution by using a calculation unit. For example, the image reconstruction unit 19 may be a computer which can analyze various data. The data analysis method (image reconstruction method) may be a filtered backprojection method, a Fourier transform method, an inverse spherical Radon transform method, or a synthetic aperture method, each method being frequently used in the photoacoustic tomography. The display device 20 may be any configuration as long as the configuration can display image data generated by the image reconstruction unit 19. For example, a liquid crystal display may be used.

When light with a plurality of wavelengths is used, an absorption coefficient distribution in a sample is calculated for each of the wavelengths by using the above-mentioned system. By comparing the obtained values with a wavelength dependence which is specific to a chromophore forming a body tissue (glucose, collagen, oxyhemoglobin, deoxyhemoglobin), imaging of a density distribution of a chromophore forming a living body can be performed.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-258569 filed Oct. 3, 2008, which is hereby incorporated by reference herein in its entirety.

1. A measuring apparatus comprising:
an acoustic transducer in which a plurality of elements are arranged, each element configured to detect an acoustic wave generated from a sample by irradiating the sample with light and convert the detected acoustic wave into an electric signal;
a movement control unit configured to move the acoustic transducer from a first position to a second position; and
a processing unit configured to generate image data on the basis of the electric signal,
wherein the acoustic transducer has a gap in the arrangement of the elements,
wherein the acoustic transducer detects an acoustic wave at the first position, is moved by the movement control unit such that the position of the gap at the first position corresponds to the position of the element at the second position, and then detects an acoustic wave at the second position, and
wherein the processing unit generates image data by merging an electric signal obtained at the first position and an electric signal obtained at the second position.

2. The measuring apparatus according to claim 1, wherein a size of the gap is an integral multiple of a size of the element, and a movement width of the acoustic transducer from the first position to the second position is an integral multiple of a width of the element in a movement direction.

3. The measuring apparatus according to claim 2, wherein the element and the gap are alternately arranged, and the movement width of the acoustic transducer from the first position to the second position is equivalent to the width of the element in the movement direction.

4. (canceled)

5. The measuring apparatus according to claim 1, wherein a light irradiation region of the sample is movable on the sample.

6. The measuring apparatus according to claim 5, wherein the acoustic transducer is moved in synchronization with the light irradiation region of the sample.

7. The measuring apparatus according to claim 1, wherein the elements in the acoustic transducer are two-dimensionally arranged.
